(19) United States
(12) Patent Application Publication
(10) Pub. No.: US 2007/0213614 A1
(43) Pub. Date: Sep. 13, 2007

(54) ULTRASONIC DIAGNOSTIC APPARATUS

(76) Inventors: Takao Suzuki, Kanagawa (JP); Hisashi Hagiwara, Kanagawa (JP); Makoto Kato, Kanagawa (JP)

Correspondence Address: MARK D. SARALINO (MEI);RENNER, OTTO, BOISSELLE & SKLAR, LLP, 1621 EUCLID AVENUE, 19TH FLOOR, CLEVELAND, OH, 44115, US

(21) Appl. No.: 11/574,140

(22) PCT Filed: Aug. 30, 2005
(86) PCT No.: PCT/JP05/15728

§ 371 Date: Feb. 23, 2007

(30) Foreign Application Priority Data
Sep. 1, 2004 (JP) 2004-253884

(51) Int. Cl. A61B 8/08

(52) U.S. Cl. 600/443

(57) ABSTRACT

An ultrasonic diagnostic apparatus according to the present invention includes: a transmitting section for driving an ultrasonic probe that transmits an ultrasonic wave toward a subject's tissue; a receiving section for receiving a reflected wave, produced by getting the ultrasonic wave reflected by the tissue of the organism, through the ultrasonic probe to generate a received signal; a plurality of filters for extracting multiple signal components, belonging to mutually different frequency bands, from the received signal; a plurality of phase detecting sections for detecting the respective phases of the multiple signal components; and a tissue tracking section for tracking the motion of the subject's tissue on the basis of the phases, thereby outputting tracking information.

### TECHNICAL FIELD

The present invention relates to an ultrasonic diagnostic apparatus for evaluating an attribute property of a subject's tissue by tracking the motion of the tissue.

### BACKGROUND ART

An ultrasonic diagnostic apparatus is used to make a noninvasive checkup on a subject by irradiating him or her with an ultrasonic wave and analyzing the information contained in its echo signal. For example, a conventional ultrasonic diagnostic apparatus that has been used extensively converts the intensity of an echo signal into its associated pixel luminance, thereby presenting the subject's structure as a tomogram. In this manner, the internal structure of the subject can be known.

Meanwhile, some people are attempting recently to track the motion of a subject's tissue more precisely and evaluate the strain and the elasticity, viscosity or any other physical (attribute) property of the tissue mainly by analyzing the phase of the echo signal.

Patent Document No. 1 discloses a method for tracking a subject's tissue highly precisely by calculating the magnitude of instantaneous displacement of a local region of the subject based on the phase difference of an ultrasonic echo signal to be transmitted and received at regular intervals and by summing the magnitudes of displacements together. Hereinafter, a method for tracking a subject's tissue as disclosed in Patent Document No. 1 will be described with reference to FIG. 162. Suppose ultrasonic pulses are transmitted toward the same location of a subject at regular intervals ΔT and received signals, generated by converting the resultant echo signals into electrical signals, are identified by y(t) and y(t+ΔT), respectively, where t represents the receiving time when the transmitting time is zero. The echo signal obtained from a measuring point, which is located at a distance x1 from the probe, and its receiving time t1 satisfy the following Equation (1):
t1=x1/(C/2) (1)
where C is the sonic velocity.

In this case, supposing the phase difference between y(t) and y(t+ΔT) is Δθ and the detection frequency is f, the magnitude of displacement Δx of the measuring point during this interval ΔT is represented by the following Equation (2):
Δx=−C·Δθ/4πf (2)

The location x1′ of the measuring point after the interval ΔT has passed is given by adding the magnitude of displacement Δx, given by Equation (2), to the original measuring point x1 as in the following Equation (3):
x1′=x1+Δx (3)

By performing this calculation repeatedly, the location of the measuring point in the subject can be tracked.

Patent Document No. 2 further develops the method of Patent Document No. 1 into a method of calculating the elasticity of a subject's tissue (e.g., an arterial vascular wall, in particular). According to this method, first, an ultrasonic wave is transmitted from a probe 101 toward the blood vessel 222 of a subject 230 as shown in FIG. 17. And the echo signals, reflected from measuring points A and B on the vascular wall of the blood vessel 222, are analyzed by the method of Patent Document No. 1, thereby tracking the motions of the measuring points A and B. FIG. 18 shows the tracking waveforms TA and TB of the measuring points A and B along with an electrocardiographic complex ECG.

As shown in FIG. 18, the tracking waveforms TA and TB have the same periodicity as the electrocardiographic complex ECG, which shows that the artery dilates and shrinks in sync with the cardiac cycle of the heart. More specifically, when the electrocardiographic complex ECG has outstanding peaks called “R waves”, the heart starts to shrink, thus pouring blood flow into the artery and raising the blood pressure. As a result, the vascular wall is dilated rapidly. That is why soon after the R wave has appeared on the electrocardiographic complex ECG, the artery dilates rapidly and the tracking waveforms TA and TB rise steeply, too. After that, however, as the heart dilates slowly, the artery shrinks gently and the tracking waveforms TA and TB gradually fall to their original levels. The artery repeats such a motion cyclically.

The difference between the tracking waveforms TA and TB is represented as a waveform W showing a variation in thickness between the measuring points A and B. Supposing the maximum variation of the thickness variation waveform is ΔW and the reference thickness between the measuring points A and B during initialization (i.e., the end of the diastole) is Ws, the magnitude of maximum strain ε between the measuring points A and B is calculated by the following Equation (4):
ε=ΔW/Ws (4)

As this strain is caused due to the difference between the blood pressures applied to the vascular wall, the elasticity Er between the measuring points A and B is given by:
Er=ΔP/ε=ΔP·Ws/ΔW (5)
where ΔP is the blood pressure difference at this time.

Therefore, by measuring the elasticity Er for multiple spots on a tomographic image, an image representing the distribution of elasticities can be obtained. If an atheroma 220 has been created in the vascular wall of the blood vessel 222 as shown in FIG. 17, the atheroma 220 and its surrounding vascular wall tissue have different elasticities. That is why if an image representing the distribution of elasticities is obtained, important information can be acquired in inspecting the attribute of the atheroma (e.g., how easily the atheroma may rupture, among other things).
Patent Document No. 1: Japanese Patent Application Laid-Open Publication No. 10-5226
Patent Document No. 2: Japanese Patent Application Laid-Open Publication No. 2000-229078

### DISCLOSURE OF INVENTION

### Problems to be Solved by the Invention

According to the conventional method of tracking a subject's tissue, however, if the magnitude of displacement of the subject's tissue during the transmission/reception interval ΔT exceeds a half of the wavelength of the ultrasonic waves, the magnitude of the displacement cannot be calculated accurately due to aliasing. Also, since the measurements can now be done on the order of several micrometers by the method described above, the results of measurements are more and more affected by noise. That is why to get measuring done as accurately as possible, the influence of noise needs to be minimized.

In order to overcome at least one of these problems of the prior art, an object of the present invention is to provide an ultrasonic diagnostic apparatus that can track the motion of a subject's tissue precisely and can obtain accurate attribute property.

### MEANS FOR SOLVING THE PROBLEMS

An ultrasonic diagnostic apparatus according to the present invention includes: a transmitting section for driving an ultrasonic probe that transmits an ultrasonic wave toward a subject's tissue; a receiving section for receiving a reflected wave, produced by getting the ultrasonic wave reflected by the tissue of the organism, through the ultrasonic probe to generate a received signal; a plurality of filters for extracting multiple signal components, belonging to mutually different frequency bands, from the received signal; a plurality of phase detecting sections for detecting the respective phases of the multiple signal components; and a tissue tracking section for tracking the motion of the subject's tissue on the basis of the phases, thereby outputting tracking information.

In one preferred embodiment, the filters include a first filter with a first center frequency and a second filter with a second center frequency that is higher than the first center frequency. The tissue tracking section includes an imperfect tracking section for tracking the motion of the subject's tissue on the basis of the phase of the signal component that has passed through the first filter and outputting imperfect tracking information and a detailed tracking section for outputting the tracking information about the subject's tissue based on the phase of the signal component that has passed through the second filter and by reference to the imperfect tracking information.

In another preferred embodiment, the tissue tracking section includes a plurality of tracking sections for tracking the motion of the subject's tissue on the basis of the phases to generate multiple pieces of tracking information and a computing section for figuring out noise-reduced tracking information based on the multiple pieces of tracking information generated by the tracking sections.

In this particular preferred embodiment, the computing section figures out at least one of the simple average and a weighted average of the multiple pieces of tracking information.

In still another preferred embodiment, the ultrasonic diagnostic apparatus further includes an amplitude measuring section for measuring the amplitude of at least one of the multiple signal components. If the amplitude is equal to or smaller than a predetermined value, the tissue tracking section generates the tracking information without using the signal component, of which the amplitude has been measured.

In yet another preferred embodiment, the transmitting section generates a signal to be transmitted that drives the ultrasonic probe so as to produce an ultrasonic wave, in which at least one of the frequency bands of the filters is boosted.

In yet another preferred embodiment, the ultrasonic diagnostic apparatus further includes a property evaluating section for evaluating an attribute property of the subject based on the tracking information.

In yet another preferred embodiment, the first filter belongs to a frequency band that passes a fundamental wave component of the ultrasonic wave and the second filter belongs to a frequency band that passes an n^{th} harmonic component (where n is an integer that is equal to or greater than two) of the ultrasonic wave.

An ultrasonic diagnostic apparatus controlling method according to the present invention is a method for controlling an ultrasonic diagnostic apparatus using the control section of the apparatus. The method includes the steps of: (A) generating a received signal based on a reflected wave by transmitting and receiving an ultrasonic wave with an ultrasonic probe, the reflected wave being produced by getting the ultrasonic wave reflected by a subject's tissue; (B) extracting multiple signal components, belonging to mutually different frequency bands, from the received signal; (C) detecting the respective phases of the multiple signal components; and (D) tracking the motion of the subject's tissue on the basis of the phases, thereby outputting tracking information.

In one preferred embodiment, the step (B) includes extracting a signal component with a first center frequency and another signal component with a second center frequency that is higher than the first center frequency. The step (D) includes the steps of: (D1) tracking the motion of the subject's tissue on the basis of the phase of the signal component that has the first center frequency and outputting imperfect tracking information and (D2) outputting the tracking information about the subject's tissue based on the phase of the signal component that has the second center frequency and by reference to the imperfect tracking information.

In another preferred embodiment, the step (D) includes the steps of (D1) tracking the motion of the subject's tissue on the basis of the phases to generate multiple pieces of tracking information and (D2) figuring out noise-reduced tracking information based on the multiple pieces of tracking information generated by tracking sections.

In this particular preferred embodiment, the step (D2) includes figuring out at least one of the simple average and a weighted average of the multiple pieces of tracking information.

In still another preferred embodiment, the method further includes the step of (E) measuring the amplitude of at least one of the multiple signal components between the steps (C) and (D). If the amplitude is equal to or smaller than a predetermined value, the step (D) includes generating the tracking information without using the signal component, of which the amplitude has been measured.

In yet another preferred embodiment, the step (A) includes transmitting an ultrasonic wave, in which at least one of the frequency bands of the filters is boosted, toward the subject.

In yet another preferred embodiment, the method further includes the step of (F) evaluating an attribute property of the subject based on the tracking information.

In yet another preferred embodiment, the signal component with the first center frequency includes a fundamental wave component of the ultrasonic wave and the signal component with the second center frequency includes an n^{th} harmonic component (where n is an integer that is equal to or greater than two) of the ultrasonic wave.

### EFFECTS OF THE INVENTION

According to the present invention, multiple signal components, belonging to mutually different frequency bands, are extracted from a received signal using filters and then analyzed, thereby defining the features of the respective signal components in terms of their frequency dependences. And by taking advantage of these features appropriately, the measuring accuracy can be improved.

### BRIEF DESCRIPTION OF DRAWINGS

FIG. 1 is a block diagram showing a first preferred embodiment of an ultrasonic diagnostic apparatus according to the present invention.

FIG. 2 schematically shows frequency characteristics that a received signal and a filter have in a conventional ultrasonic diagnostic apparatus.

FIG. 3 schematically shows frequency characteristics that a received signal and a filter have in another conventional ultrasonic diagnostic apparatus.

FIG. 4 schematically shows frequency characteristics that a received signal and filters may have in the ultrasonic diagnostic apparatus shown in FIG. 1.

FIG. 5 is a flowchart showing a method for controlling the ultrasonic diagnostic apparatus shown in FIG. 1.

FIG. 6 illustrates a picture that may be presented on the monitor of the ultrasonic diagnostic apparatus shown in FIG. 1.

FIG. 7 schematically shows other frequency characteristics that a received signal and filters may have in the ultrasonic diagnostic apparatus shown in FIG. 1.

FIG. 8 schematically shows other frequency characteristics that a received signal and filters may have in the ultrasonic diagnostic apparatus shown in FIG. 1.

FIG. 9 is a block diagram showing a second preferred embodiment of an ultrasonic diagnostic apparatus according to the present invention.

FIG. 10 is a flowchart showing a method for controlling the ultrasonic diagnostic apparatus shown in FIG. 9.

FIG. 11 is a block diagram showing a third preferred embodiment of an ultrasonic diagnostic apparatus according to the present invention.

FIG. 12 schematically shows frequency characteristics that a received signal and filters may have in the ultrasonic diagnostic apparatus shown in FIG. 11.

FIG. 13 is a flowchart showing a method for controlling the ultrasonic diagnostic apparatus shown in FIG. 11.

FIG. 14 is a block diagram showing a fourth preferred embodiment of an ultrasonic diagnostic apparatus according to the present invention.

FIG. 15 is a flowchart showing a method for controlling the ultrasonic diagnostic apparatus shown in FIG. 14.

FIG. 16 shows how to track a tissue based on the phase difference of an ultrasonic echo signal.

FIG. 17 is a schematic representation showing how to calculate the elasticity of a vascular wall.

FIG. 18 shows how to calculate the magnitude of strain using a tracking waveform obtained from a vascular wall.

### DESCRIPTION OF REFERENCE NUMERALS

100 control section
101 probe
102 transmitting section
103 receiving section
104 tomographic image processing section
106 image synthesizing section
107 monitor
113A, 113B, 113X band-pass filter
114A, 114B, 114X phase detecting section
115 tissue imperfect tracking section
116 tissue detailed tracking section
117 tissue property evaluating section
118 amplitude measuring section
119 tissue detailed tracking section
121A, 121B, 121X tissue tracking section
122 computing section
123 computing section
171, 172, 173, 174 tissue tracking section
200 tomogram
201 tissue property image
202 tomographic reflection intensity scale
203 tissue property image scale
204 biomedical signal waveform
301, 302, 303, 304 ultrasonic diagnostic apparatus

### BEST MODE FOR CARRYING OUT THE INVENTION

### Embodiment 1

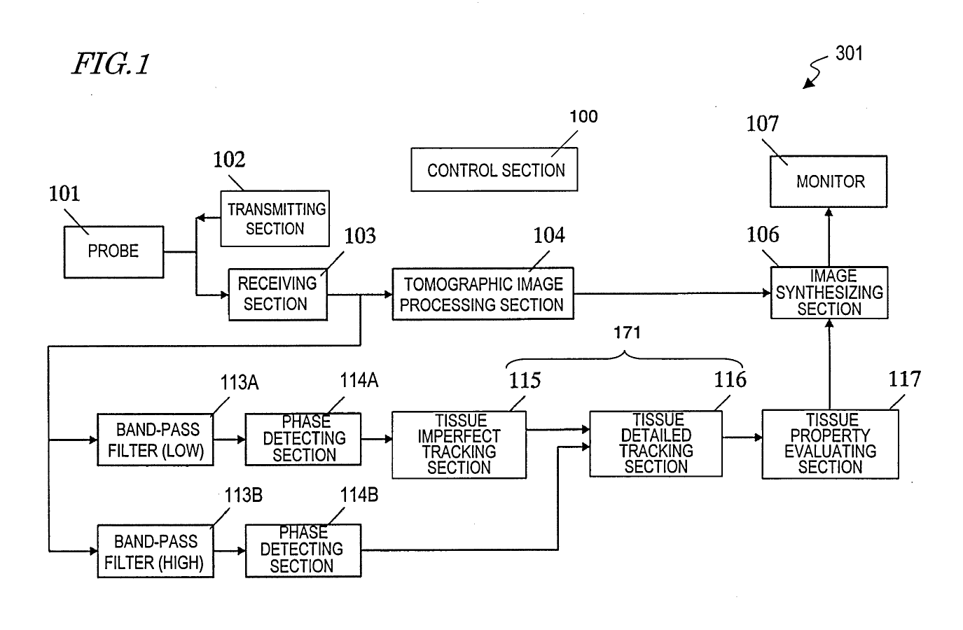

FIG. 1 is a block diagram showing a first preferred embodiment of an ultrasonic diagnostic apparatus according to the present invention. As shown in FIG. 1, the ultrasonic diagnostic apparatus 301 includes a transmitting section 102, a receiving section 103, a tomographic image processing section 104, band-pass filters 113A, 113B, phase detecting sections 114A, 114B, a tissue tracking section 171, a tissue property evaluating section 117 and an image synthesizing section 106. The apparatus 301 also includes a control section 100 for controlling all of these elements. Although not shown in FIG. 1, an input device such as a keyboard, a track ball, a switch, a button or a key and an output device such as an LCD monitor are also connected to the control section 100. The control section 100 includes an ASIC, an FPGA, a DSP, a CPU, a memory and so on, stores a program for performing respective processing steps for controlling the ultrasonic diagnostic apparatus 301 and reads and executes the program if necessary.

In accordance with the instruction given by the control section 100, the transmitting section 102 generates a high-voltage signal that drives the probe 101 at a specified timing. The probe 101 converts the signal that has been generated by the transmitting section 102 into an ultrasonic wave and sends out the ultrasonic wave toward a subject, and also detects an ultrasonic echo that has been reflected by an internal organ of the subject and converts the echo into an electrical signal. A number of piezoelectric transducers are arranged in the probe 101. By changing the piezoelectric transducers to use, the timing to apply a voltage to the piezoelectric transducers, or the voltages themselves, the probe 101 controls the scan line position, angle of deflection and focus of the ultrasonic waves to transmit and receive.

The receiving section 103 amplifies the received signal, and adds appropriate delays to the signals received from the respective piezoelectric transducers. In this manner, the receiving section 103 detects either only an ultrasonic wave that has been reflected from a predetermined point (i.e., a focused ultrasonic beam) or only an ultrasonic wave that has come from a predetermined direction (or at a predetermined angle of deflection). In the latter case, the receiving section 103 forms an ultrasonic beam so to speak.

The tomographic image processing section 104 includes a filter, a detector, a logarithmic amplifier and a scanning converter, and analyzes mainly the amplitude of the received signal, thereby presenting the internal structure of the subject as an image.

The band-pass filters 113A and 113B have mutually different pass bands and extract signal components in their respective pass bands from the received signal supplied from the receiving section. The center frequencies f1 and f2 of the respective pass bands of the band-pass filters 113A and 113B satisfy the inequality f1<f2. That is why the signal that has passed the band-pass filter 113A includes the low-frequency components of the received signal, while the signal that has passed the band-pass filter 113B includes the high-frequency components of the received signal.

As will be described in detail later, by getting multiple signal components in mutually different frequency bands extracted from the received signal by the band-pass filters 113A and 113B and by analyzing those signal components, their respective features can be obtained from the signal components in terms of frequency dependence. Thus, by taking advantage of these features appropriately, the measuring accuracy can be increased.

The phase detecting sections 114A and 114B may be implemented as orthogonal detectors, for example, and detect the phases of the signal components of the received signal, of which the bands have been limited by the band-pass filters 113A and 113B.

The tissue tracking section 171 includes a tissue imperfect tracking section 115 and a tissue detailed tracking section 116, and tracks the motion of the subject's tissue on the basis of the phases of the signal components that have been detected by the phase detecting sections 114A and 114B by using Equations (2) and (3), thereby outputting tracking information. More specifically, the tissue imperfect tracking section 115 generates the tracking information on the basis of the phase of the signal component that has been detected by the phase detecting section 114A by using Equations (2) and (3). As will be described later, this tracking information has been generated based on the low frequency components of the received signal, and therefore, is imperfect tracking information with a low resolution. The tracking information includes a tracking waveform representing a variation in the phase of the received signal at a measuring point, the magnitude of motion of the measuring point, and the positional displacement of the measuring point.

The tissue detailed tracking section 116 generates and outputs the detailed tracking information of each tissue on the basis of the phase of the signal component that has been detected by the phase detecting section 114B and the imperfect tracking information provided by the tissue imperfect tracking section 115 by using Equations (2) and (3).

The tissue property evaluating section 117 receives the detailed tracking information from the tissue tracking section 171, calculates a parameter representing the tissue's property such as strain factor, magnitude of strain, elasticity or viscosity, and outputs it as a numerical value, a two-dimensional image, or a sound. In calculating the elasticity or viscosity, the tissue property evaluating section 117 receives information about the stress that has caused a kinetic variation in the subject's tissue from an external device. If the subject's tissue is the arterial vascular wall, then the tissue property evaluating section 117 receives the systolic and diastolic blood pressure values from a blood pressure manometer, for example, and figures out the parameter using Equation (5).

The image synthesizing section 106 synthesizes together the tomographic image supplied from the tomographic image processing section 104, the image or the numerical value representing the tissue's property as provided by the tissue property evaluating section, and other numerical parameters and presents them on the monitor 107. The ultrasonic diagnostic apparatus 301 may include a dedicated monitor 107 for that purpose or use a general computer monitor.

Next, the operations of the band-pass filters 113A, 113B, the phase detecting sections 114A, 114B and tissue tracking section 171, which form the core of the present invention, will be described in detail.

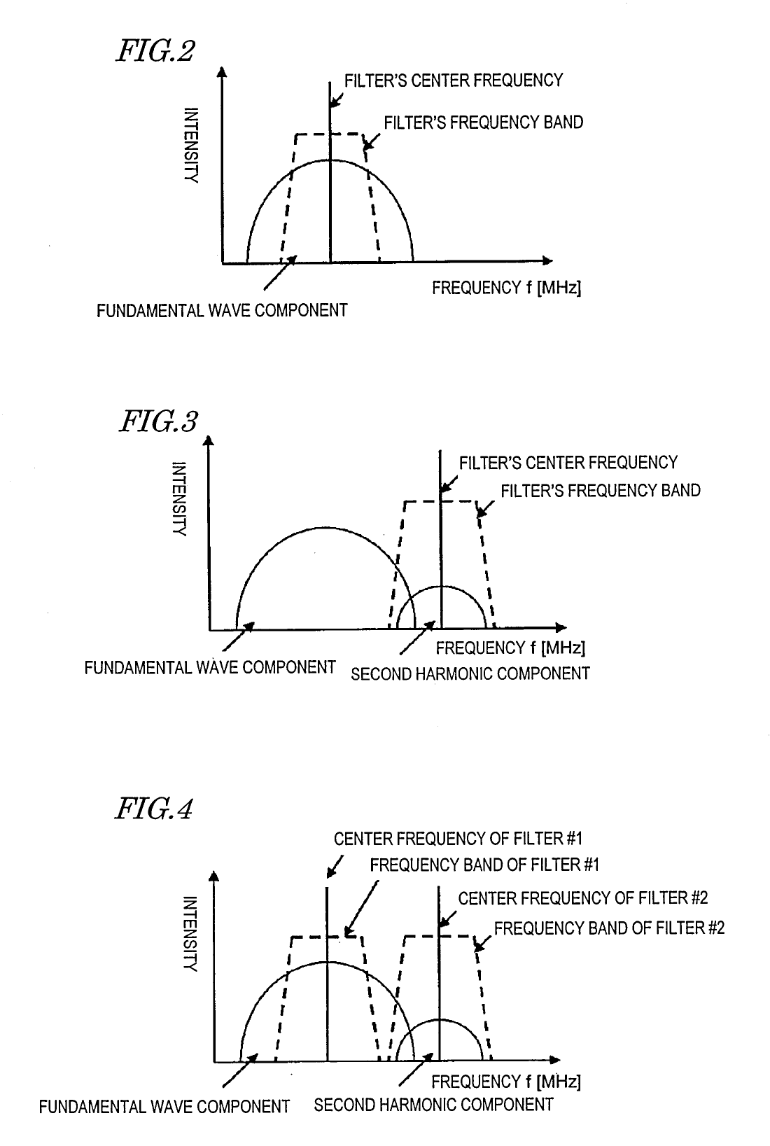

FIGS. 2 and 3 schematically show frequency characteristics that band-pass filters and received signals may have in conventional ultrasonic diagnostic apparatuses. As shown in FIG. 2, a conventional ultrasonic diagnostic apparatus gets a fundamental wave component of the received signal selectively extracted by the filter from around the center frequency (or transmission frequency) thereof and performs the calculations by Equations (2) and (3) on the basis of the phase of the extracted signal component, thereby tracking the motion of the subject's tissue. It is known that if an ultrasonic wave has been sent out toward a subject, the variations caused by the ultrasonic wave in the subject's tissue have non-linearity and the reflected wave includes a harmonic component. Thus, it is known that to detect that harmonic component, the pass band of the filter may be doubled compared to the transmission frequency, the second harmonic component generated by the subject's tissue may be extracted and the motion of the subject's tissue may be tracked on the basis of the phase and Equations (2) and (3) as shown in FIG. 3.

The higher the frequency of the ultrasonic wave to be detected, the higher the resolution and the more precisely the subject's tissue can be tracked. However, as the frequency rises, the influence of aliasing increases and it becomes more and more difficult to keep up with quick motions. That is to say, the phase of the received signal can no longer be defined unambiguously due to aliasing. For example, even if a phase of −π/2 has been detected by the phase detecting section 114B, it cannot be determined whether this phase is really −π/2 or has been detected erroneously as −π/2 due to aliasing, although it is actually 3π/2. As can be seen, a tradeoff is inevitable between the tracking precision and the ability to keep up with the motion velocity.

FIG. 4 schematically shows the frequency characteristics of the band-pass filters 113A, 113B and their received signals that are used in the ultrasonic diagnostic apparatus 301 of the present invention. To extract only the fundamental wave component from the received signal, the center frequency f1 of the pass band of the band-pass filter 113A is set equal to that of the fundamental wave component. And its bandwidth is also defined so as to include only the fundamental wave component. On the other hand, to extract only the second harmonic component from the received signal, the center frequency f2 of the pass band of the band-pass filter 113B is set equal to that of the second harmonic component of the received signal. And its bandwidth is also defined so as to include only the second harmonic component. The second harmonic component in the received signal may either have been generated due to the non-linearity of the subject's tissue or be included beforehand in the ultrasonic wave to be transmitted by the transmitting section 103. For example, by using a pulsed transmitted wave, the harmonics can be generated in the transmitted wave and the second harmonic component of the received signal may be included beforehand.

The phase detecting section 114A detects the phase of the fundamental wave component in the received signal. The tissue imperfect tracking section 115 generates tracking information, including a tracking waveform that represents the variation in the phase of the fundamental wave component in the received signal, the magnitude of motion of the measuring point, and the positional displacement of the measuring point, on the basis of the phase detected and by using Equations (2) and (3). At the frequencies of the fundamental wave component, no aliasing is produced and the motion of the subject's tissue can be detected properly even if the tissue is moving at high velocities. That is why these pieces of tracking information may contain some error depending on the resolution determined by the frequency but still show accurate results of measurements.

The phase detecting section 114B detects the phase of the second harmonic component in the received signal. The tissue detailed tracking section 116 generates tracking information, including a tracking waveform that represents the variation in the phase of the fundamental wave component in the received signal, the magnitude of motion of the measuring point, and the positional displacement of the measuring point, on the basis of the phase detected and by using Equations (2) and (3). In this case, as the second harmonic components include high frequencies, the tracking could be affected by aliasing as described above. To eliminate the influence of such aliasing, the tissue detailed tracking section 116 receives the tracking information from the tissue imperfect tracking section 115. The tracking information received does not have high resolution but has an accurate value. That is why even if the phase of the second harmonic component cannot be defined due to aliasing, the phase of the second harmonic component can be determined properly according to the tracking information received from the tissue imperfect tracking section 115. After that, the tracking information, including a tracking waveform that represents the magnitude of motion of the measuring point and the positional displacement of the measuring point, is generated by using Equations (2) and (3).

A specific example will be described in terms of numerical values. Suppose the sonic velocity is 1,540 m/s, the fundamental frequency is 5 MHz, and the second harmonic is 10 MHz. In that case, if the magnitude of motion of the measuring point set on the subject is 26 μm, no aliasing will be produced at the fundamental frequency or the second harmonic. That is why at the fundamental frequency, the phase is detected to be −π/3 and the magnitude of motion of 26 μm, containing a certain amount of error, can be figured out based on this phase by Equation (2). At the second harmonic, on the other hand, the phase is detected to be −2π/3 and the magnitude of motion of 26 μm can also be figured out by Equation (2) with the error reduced.

On the other hand, if the magnitude of motion of the measuring point set on the subject is 52 μm, no aliasing will be produced at the fundamental frequency but aliasing will be produced at the second harmonic. That is why at the fundamental frequency, the phase is detected to be −2π/3 and the magnitude of motion of 52 μm, containing a certain amount of error, can be figured out based on this phase by Equation (2). At the second harmonic, on the other hand, the phase should be detected to be −4 π/3 but is actually detected as 2 π/3 and the magnitude of motion of −26 μm is figured out by Equation (2).

In that case, according to the present invention, first, the tissue imperfect tracking section 115 measures the fundamental wave and figures out the magnitude of motion of about 52 μm. The tissue detailed tracking section 116 receives, from the tissue imperfect tracking section 115, the tracking information including a magnitude of motion of about 52 μm. Thus, it is determined that the phase detected by the phase detecting section 114B is not 2/3π but is −4π/3. And the magnitude of motion of 52 μm can be figured out based on this phase with the error reduced. Since the second harmonic component that has higher frequencies than the fundamental wave component is used, the calculations made by the tissue detailed tracking section 116 have higher resolution and higher precision. Consequently, the tracking information generated by the tissue detailed tracking section 116 has high precision.

In addition, if the harmonic component produced by a nonlinear phenomenon of the subject's tissue is used, then the influences of various artifacts such as side lobe and multiple echoes can be reduced, too.

FIG. 5 is a flowchart showing a method for controlling the ultrasonic diagnostic apparatus 301. As already described with reference to FIG. 1, the ultrasonic diagnostic apparatus 301 carries out the measurements by getting the respective elements thereof controlled by its own control section 100. More specifically, first, the transmitting section 102 transmits an ultrasonic wave toward the subject by driving the ultrasonic probe 101 and the receiving section 103 receives the reflected wave from the subject by way of the probe 101. In this manner, the receiving section 103 generates a received signal based on the wave that has been reflected from the subject's tissue (in Step S501).

Next, the band-pass filters 113A and 113B extract a plurality of signal components belonging to mutually different frequency bands (i.e., a fundamental wave component and a second harmonic component) from the received signal (in Step S502). The phases of the signal components extracted are detected by the phase detecting sections 114A and 114B, respectively (in Step S503). Then, the tissue imperfect tracking section 115 obtains the tracking information of the subject's tissue on the basis of the phase of the signal component detected by the phase detecting section 114A (in Step S504).

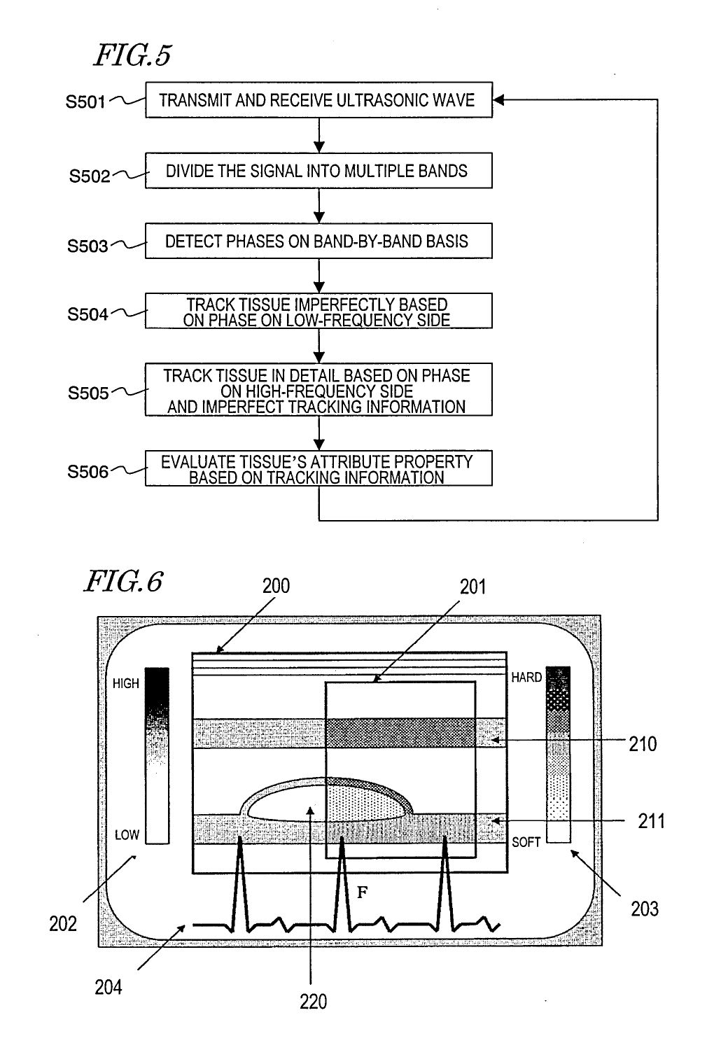

The tissue detailed tracking section 116 obtains the detailed tracking information of the subject's tissue in accordance with the tracking information provided by the tissue imperfect tracking section 115 and on the basis of the phase of the signal component detected by the phase detecting section 114B (in Step S505). The tissue property evaluating section 117 evaluates the attribute property of the tissue based on the detailed tracking information of the subject's tissue (in Step S506). By repeatedly performing this procedure, the location of each tissue of the subject can be tracked sequentially. FIG. 6 illustrates a picture that may be presented on the monitor 107 and shows an exemplary result of measurement of the vascular wall's elasticity. In FIG. 6, a two-dimensional elasticity image 201, representing the distribution of elasticity at a site on the vascular wall in colors, is superimposed on a vascular wall's monochrome tomographic image 200 obtained by the tomographic image processing section 104 on the monitor. The two-dimensional elasticity image 201 sets the areas to monitor so as to include the vascular anterior and posterior walls 210 and 211.

In transmitting or receiving the ultrasonic wave, the monochrome tomographic image 200 is updated at a rate of 10 frames per second as in a conventional ultrasonic diagnostic apparatus. Meanwhile, the elasticity image 201 is updated once every cardiac cycle. The monochrome tomographic image 200 is displayed at monochromatic gray scales corresponding to the reflection intensities along with a scale 202 indicating the reflection intensities. On the other hand, the elasticity image 201 is displayed in color tones corresponding to the elasticity values along with a scale 203 indicating the elasticity values. Also displayed under the monochrome tomographic image 200 is a biomedical signal waveform 204 such as an electrocardiogram.

FIG. 6 schematically shows the posterior wall 211 with an atheroma 220. The ultrasonic diagnostic apparatus 301 of this preferred embodiment can track the location of a subject's tissue with high precision as described above, and therefore, can figure out elasticity accurately. That is why the elasticity distribution of the atheroma 220 produced on the vascular wall can be obtained and important information to determine an attribute of the atheroma 220 (e.g., how easily the atheroma may rupture, among other things) can be acquired with high precision. As described above, the ultrasonic diagnostic apparatus of this preferred embodiment gets a signal representing a fundamental wave component and a signal representing a second harmonic component extracted from a received signal by filters. The tissue imperfect tracking section analyzes the signal representing the fundamental wave component, thus obtaining accurate tracking information that is never affected by aliasing even if the subject's tissue is moving at high velocities. Also, the tissue detailed tracking section analyzes the signal representing the second harmonic component. Even if aliasing is produced in this case, accurate tracking information can be acquired based on the tracking information obtained by the tissue imperfect tracking section. Consequently, the ultrasonic diagnostic apparatus of this preferred embodiment can track the quickly moving subject's tissue highly precisely. As a result, the elasticity distribution of the arterial vascular wall can be measured highly accurately.

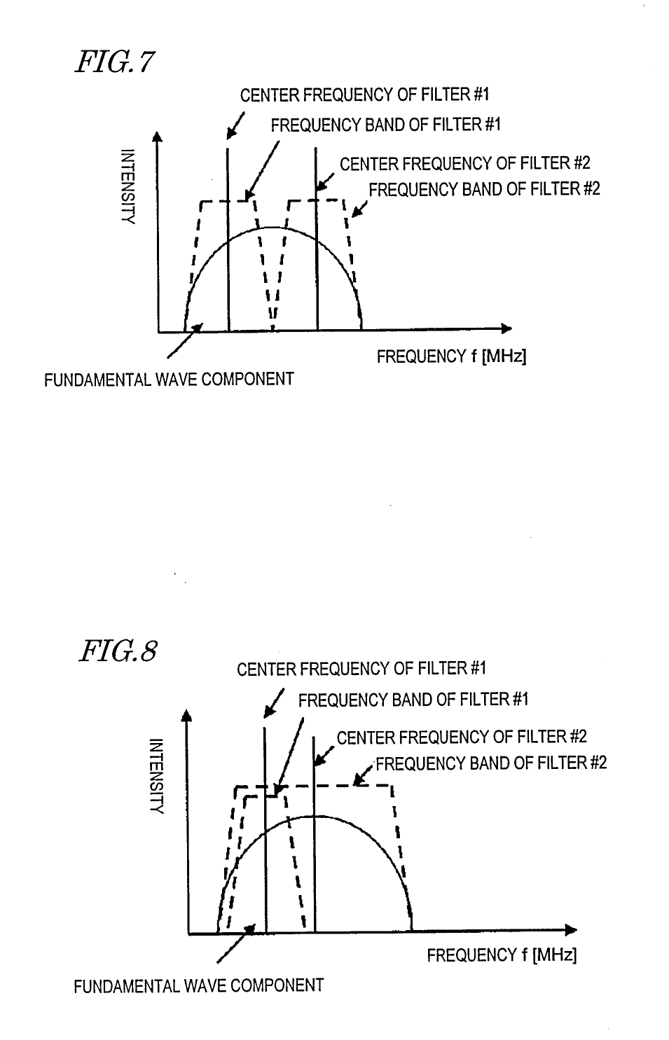

In the preferred embodiment described above, the band-pass filters 113A and 113B are used to extract a fundamental wave component and a second harmonic component, respectively, from the received signal. However, the band-pass filters 113A and 113B may have other band-pass characteristics. For example, the band-pass filter 113A may extract a relatively low frequency component from the fundamental wave components of the received signal, while the band-pass filter 113B may extract a relatively high frequency component from the fundamental wave components of the received signal as shown in FIG. 7. The low frequency component does not have to be the relatively low frequency component of the fundamental wave but may also be a low frequency component generated due to the non-linearity of the subject's tissue or included beforehand in the ultrasonic wave to transmit.

Even in that case, the tissue imperfect tracking section 115 analyzes the low frequency component by Equations (2) and (3), thereby acquiring imperfect tracking information as in the example described above. Next, the tissue detailed tracking section 119 analyzes the high frequency component based on the imperfect tracking information, thereby tracking the motion of the subject's tissue in detail. As a result, compared to carrying out measurements using the conventional filter shown in FIG. 2, a quicker motion of the subject's tissue can be kept up with by using the low frequency component and tracking can be done more precisely by using the high frequency component.

Still alternatively, the band-pass filter 113A may extract a relatively low frequency component in a narrower band from the fundamental wave components of the received signal, while the band-pass filter 113B may extract the entire fundamental wave components from the received signal as shown in FIG. 8. The band-pass filter 113B has a pass band that partially overlaps with, but is broader than, that of the band-pass filter 113A. That is why the band-pass filter 113B may be regarded as extracting a higher frequency component than the band-pass filter 113A. Even by using the band-pass filters 113A and 113B with such frequency characteristics, a quicker motion of the subject's tissue can be kept up with by using the low frequency component and tracking can be done more precisely by using the high frequency component, compared to carrying out measurements using the conventional filter shown in FIG. 2. On top of that, as the high frequency component has a broader bandwidth, a higher resolution is realized.

Furthermore, in the preferred embodiment described above, two band-pass filters are used to extract signal components belonging to two different frequency bands from the received signal. However, the number of signal components to be extracted does not have to be two but may also be three or more. Also, the frequency bands of the signal components to be extracted by the band-pass filters just may not be quite equal to, but may partially overlap with, each other. An even quicker motion of the subject's tissue can be kept up with by using a lower frequency component of the received signal. And tracking can be done even more precisely by using a higher frequency component of the received signal.

Furthermore, in the preferred embodiment described above, a second harmonic component is extracted. Alternatively, an n^{th}-order harmonic component (where n is an integer that is equal to or greater than three) may be extracted to acquire the tracking information. Optionally, a transmitted signal in which at least one of the signal components extracted by the band-pass filters is boosted may be used such that the at least one of the signal components extracted by the band-pass filters can be detected with predetermined amplitude.

### Embodiment 2

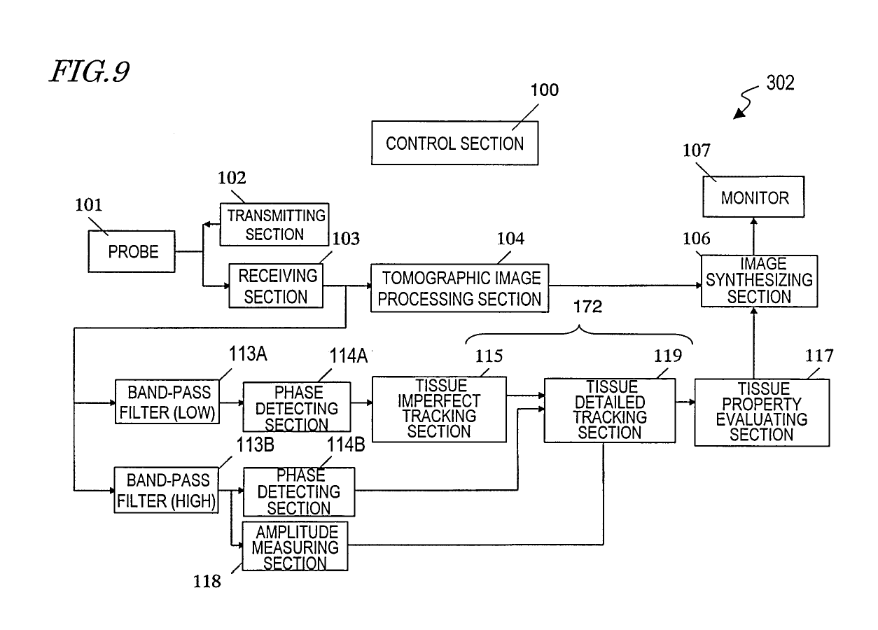

FIG. 9 is a block diagram showing a second preferred embodiment of an ultrasonic diagnostic apparatus according to the present invention. As shown in FIG. 9, the ultrasonic diagnostic apparatus 302 further includes an amplitude measuring section 118, which is not included in the ultrasonic diagnostic apparatus 301 of the first preferred embodiment described above.

The amplitude measuring section 118 measures the amplitude of the second harmonic component that has been extracted by the band-pass filter 113B from the received signal. If the amplitude is equal to or smaller than a predetermined threshold value, the amplitude measuring section 118 generates a signal representing that amplitude and outputs it to the tissue detailed tracking section 119. In response to that signal supplied from the amplitude measuring section 118, the tissue detailed tracking section 119 does not generate the tracking information based on the second harmonic component but outputs as it is the tracking information provided by the tissue imperfect tracking section 115.

If either the received signal itself or the signal component extracted from the received signal has small amplitude, the signal or its component has been affected by noise seriously and has a small SNR. Therefore, even if the phase is detected using such a signal, the precision is too low to make accurate measurements.

According to this preferred embodiment, by not generating the tracking information from the received signal or a signal component extracted from it in that case, the decrease in tracking precision is avoided.

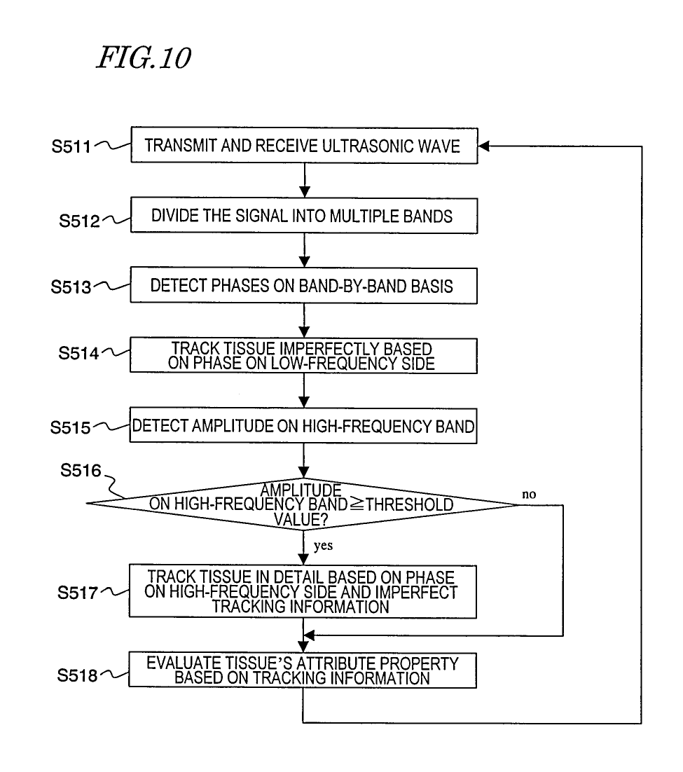

FIG. 10 is a flowchart showing a method for controlling the ultrasonic diagnostic apparatus 302. Hereinafter, a method for controlling the ultrasonic diagnostic apparatus 302 will be described with reference to FIGS. 9 and 10. First, the transmitting section 102 transmits an ultrasonic wave toward the subject by driving the ultrasonic probe 101 and the receiving section 103 receives the reflected wave from the subject by way of the probe 101. In this manner, the receiving section 103 generates a received signal based on the wave that has been reflected from the subject's tissue (in Step S511).

Next, the band-pass filters 113A and 113B extract a plurality of signal components belonging to mutually different frequency bands from the received signal (in Step S512). The phases of the signal components extracted are detected by the phase detecting sections 114A and 114B, respectively (in Step S513). Then, the tissue imperfect tracking section 115 obtains the tracking information of the subject's tissue on the basis of the phase of the signal component detected by the phase detecting section 114A (in Step S514).

The amplitude measuring section 118 measures the amplitude of the signal component extracted by the band-pass filter 113B (in Step S514). In Step S516, if the amplitude is equal to or greater than a predetermined threshold value, the tissue detailed tracking section 119 obtains the detailed tracking information of the subject's tissue in accordance with the tracking information provided by the tissue imperfect tracking section 115 and on the basis of the phase of the signal component detected by the phase detecting section 114B (in Step S517). On the other hand, if the amplitude is smaller than the predetermined threshold value in Step S516, then the tissue detailed tracking section 116 does not use the signal component extracted by the band-pass filter 113B but outputs as it is the tracking information provided by the tissue imperfect tracking section 115.

The tissue property evaluating section 117 evaluates the attribute property of the tissue based on the tracking information provided by the tissue detailed tracking section 116 (in Step S518). By repeatedly performing this procedure, the location of each tissue of the subject can be tracked sequentially.

### Embodiment 3

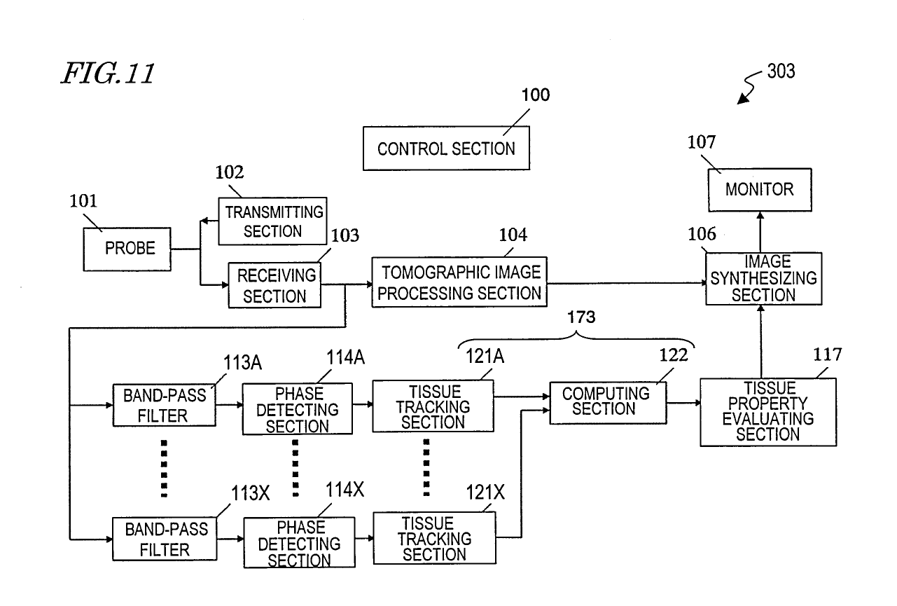

FIG. 11 is a block diagram showing a third preferred embodiment of an ultrasonic diagnostic apparatus according to the present invention. As shown in FIG. 11, the ultrasonic diagnostic apparatus 303 further includes a plurality of band-pass filters 113A through 113X, a plurality of phase detecting sections 114A through 114X and a tissue tracking section 173 unlike the ultrasonic diagnostic apparatus 301 of the first preferred embodiment described above.

The band-pass filters 113A through 113X extract signal components belonging to mutually different frequency bands from the received signal. The phase detecting sections 114A through 114X detect the phases of those signal components belonging to the mutually different frequency bands.

The tissue tracking section 173 includes tissue tracking sections 121A through 121X and a computing section 122. Each of the tissue tracking sections 121A through 121X acquires the tracking information of the subject's tissue based on the phase detected and by using Equations (2) and (3). If there is no noise, the same tracking information should be acquired by the tissue tracking sections 121A through 121X. If there is noise, however, the noise level will be different from one frequency band to another, thus causing an error in the tracking waveform.

The computing section 122 generates noise-reduced tracking information based on multiple pieces of tracking information provided by the respective tissue tracking sections 121A through 121X. More specifically, the computing section 122 calculates either a simple average or a weighted average of those pieces of tracking information provided by the respective tissue tracking sections 121A through 121X, thereby outputting averaged tracking information. The weighted average may be calculated by giving the heaviest weight to a frequency band around the center frequency of the transmitted or received waveform and decreasing weights to its surrounding bands. Alternatively, the average may also be calculated with some of those pieces of tracking information, provided by the tissue tracking sections 121A through 121X, excluded if their values are far different from the others.

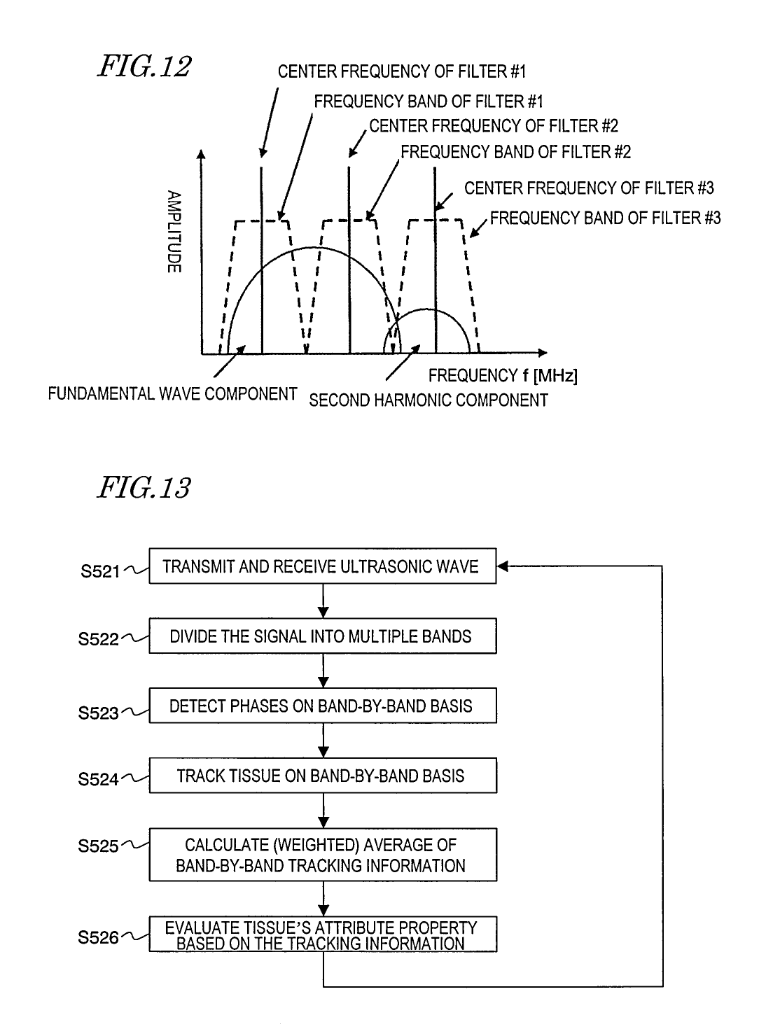

FIG. 12 shows exemplary pass band characteristics and the frequency characteristics of the received signal in a situation where three band-pass filters are used. By performing tracking and averaging using multiple frequency bands in this manner, the influence of noise can be reduced and tracking can be carried out more accurately. Also, the noise superposed on the received signal does not affect equally the entire frequency band of the received signal. If multiple signal components belonging to mutually different frequency bands are extracted from the received signal, some of the signal components have been affected by noise heavily but others only lightly. Those signal components that are heavily affected by the noise should have pieces of tracking information that are far apart from those of the other signal components. That is why by calculating an average with those signal components excluded, tracking can be carried out highly accurately with the influence of noise further reduced.

Also, if low frequency components are extracted from the received signal as the pass bands of the band-pass filters 113A through 113X and are used to carry out tracking, an even quicker motion of the subject's tissue can be kept up with. On the other hand, if high frequency components are extracted from the received signal, tracking can be carried out more precisely.

FIG. 13 is a flowchart showing a method for controlling the ultrasonic diagnostic apparatus 303. Hereinafter, a method for controlling the ultrasonic diagnostic apparatus 303 will be described with reference to FIGS. 11 and 13. First, the transmitting section 102 transmits an ultrasonic wave toward the subject by driving the ultrasonic probe 101 and the receiving section 103 receives the reflected wave from the subject by way of the probe 101. In this manner, the receiving section 103 generates a received signal based on the wave that has been reflected from the subject's tissue (in Step S521).

Next, the band-pass filters 113A through 113X extract a plurality of signal components belonging to mutually different frequency bands from the received signal (in Step S522). The phases of the signal components extracted are detected by the phase detecting sections 114A through 114X, respectively (in Step S523). Then, the tissue tracking sections 121A through 121X acquire the tracking information on the basis of the phases detected (in Step S524).

The computing section 123 calculates the average of those pieces of tracking information provided by the respective tissue tracking sections 121A through 121X (in Step S525). The tissue property evaluating section 117 evaluates the attribute property of the tissue based on the tracking information provided by the computing section 122 (in Step S526). By repeatedly performing this procedure, the location of each tissue of the subject can be tracked sequentially.

### Embodiment 4

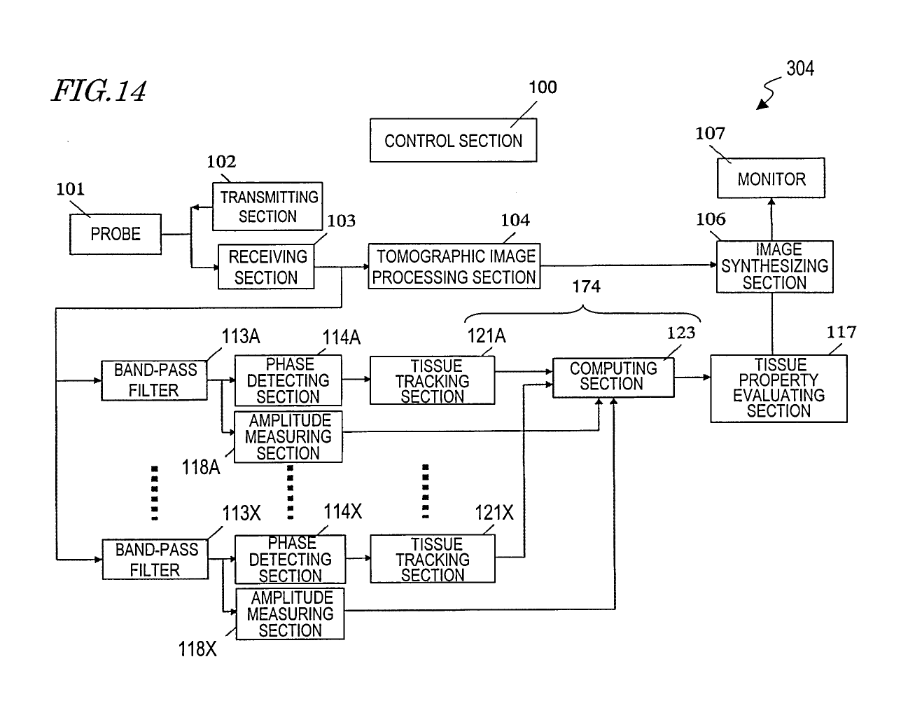

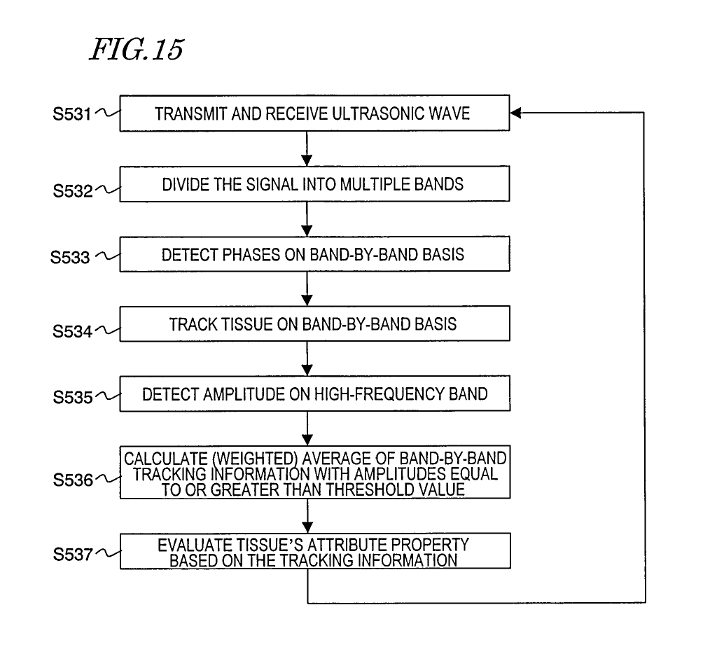

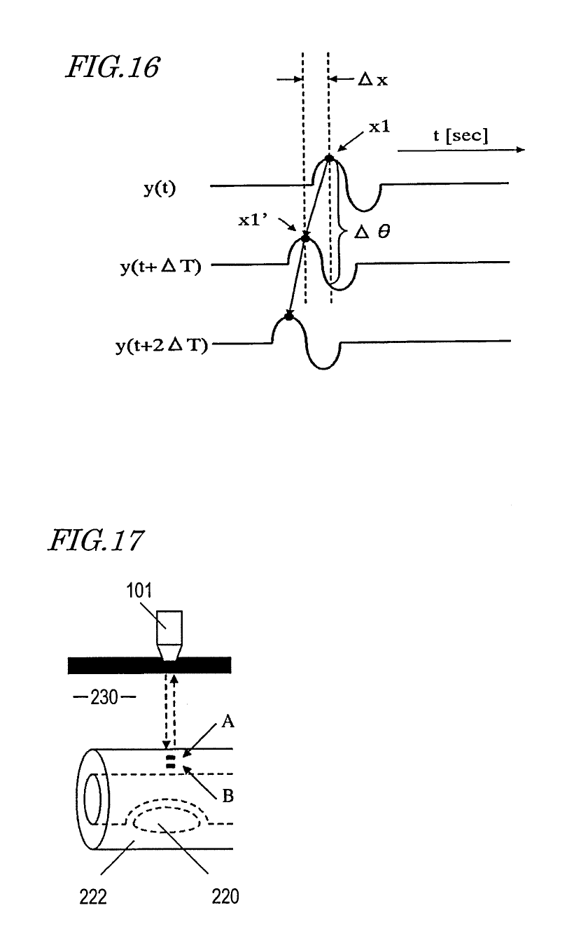

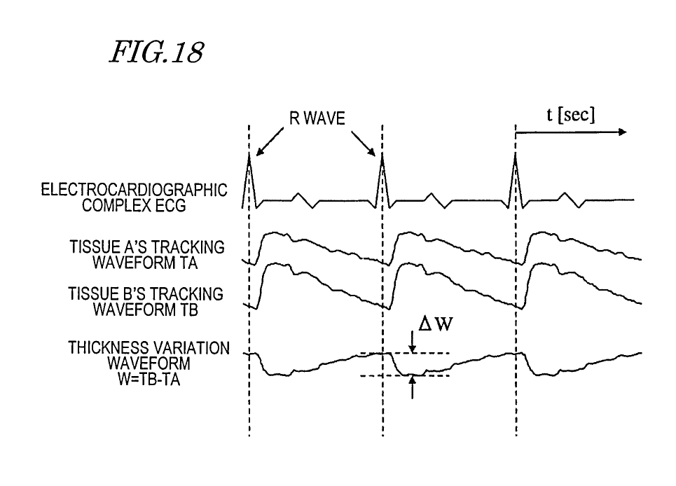

FIG. 14 is a block diagram showing a fourth preferred embodiment of an ultrasonic diagnostic apparatus according to the present invention. As shown in FIG. 14, the ultrasonic diagnostic apparatus 304 further includes amplitude measuring sections 118A through 118X, which are not included in the ultrasonic diagnostic apparatus 303 of the third preferred embodiment described above.

The amplitude measuring sections 118A through 118X measure the amplitudes of the signal components that have been extracted by the band-pass filters 113A through 113X from the received signal. If the amplitude is equal to or smaller than a predetermined threshold value, the amplitude measuring sections 118A through 118X generate a signal representing that amplitude and output it to the computing section 123. On receiving that signal from any of the amplitude measuring sections 118A through 118X, the computing section 123 excludes the piece of tracking information, obtained from the associated signal component, from the object of averaging and calculates the average of the other pieces of tracking information. As already described for the third preferred embodiment, various averaging methods may be used. Optionally, a weighted average may be calculated according to the amplitudes.

If the signal component extracted from the received signal has small amplitude, the signal component has been affected by noise seriously and has a small SNR. Therefore, even if the phase of the tracking information is detected using such a signal, the precision is too low to make accurate measurements. According to this preferred embodiment, by not generating the tracking information from the received signal or a signal component extracted from it in that case, the decrease in tracking precision is avoided.

FIG. 15 is a flowchart showing a method for controlling the ultrasonic diagnostic apparatus 304. Hereinafter, a method for controlling the ultrasonic diagnostic apparatus 303 will be described with reference to FIGS. 14 and 15. First, the transmitting section 102 transmits an ultrasonic wave toward the subject by driving the ultrasonic probe 101 and the receiving section 103 receives the reflected wave from the subject by way of the probe 101. In this manner, the receiving section 103 generates a received signal based on the wave that has been reflected from the subject's tissue (in Step S531).

Next, the band-pass filters 113A through 113X extract a plurality of signal components belonging to mutually different frequency bands from the received signal (in Step S532). The phases of the signal components extracted are detected by the phase detecting sections 114A through 114X, respectively (in Step S533). Then, the tissue tracking sections 121A through 121X acquire the tracking information on the basis of the phases detected (in Step S534). Furthermore, the amplitude measuring sections 118A through 118X detect the amplitudes of the respective signal components (in Step S535).

The computing section 122 calculates the average of those pieces of tracking information provided by the respective tissue tracking sections 121A through 121X (in Step S536). In this processing step, the computing section 123 receives the amplitude values of the respective signal components from the amplitude measuring sections 118A through 118X. If the amplitude is smaller than a predetermined threshold value, then the computing section 123 does not use the piece of tracking information obtained from the signal component in calculating the average. The tissue property evaluating section 117 evaluates the attribute property of the tissue based on the tracking information provided by the computing section 123 (in Step S537). By repeatedly performing this procedure, the location of each tissue of the subject can be tracked sequentially.

### INDUSTRIAL APPLICABILITY

The present invention is preferably implemented as an ultrasonic diagnostic apparatus for tracking the motion of a subject's tissue. Among other things, the present invention can be used particularly effectively as an ultrasonic diagnostic apparatus for evaluating the attribute property of a tissue (such as the elasticity of the arterial vascular wall of an organism).

1. An ultrasonic diagnostic apparatus comprising:
a transmitting section for driving an ultrasonic probe that transmits an ultrasonic wave toward a subject's tissue;
a receiving section for receiving a reflected wave through the ultrasonic probe to generate a received signal, the reflected wave being produced by getting the ultrasonic wave reflected by the tissue of the organism;
a plurality of filters for extracting multiple signal components, belonging to mutually different frequency bands, from the received signal;
a plurality of phase detecting sections for detecting the respective phases of the multiple signal components; and
a tissue tracking section for tracking the motion of the subject's tissue on the basis of the phases, thereby outputting tracking information.

2. The ultrasonic diagnostic apparatus of claim 1, wherein the filters include a first filter with a first center frequency and a second filter with a second center frequency that is higher than the first center frequency,
wherein the tissue tracking section includes: an imperfect tracking section for tracking the motion of the subject's tissue on the basis of the phase of the signal component that has passed through the first filter and outputting imperfect tracking information; and a detailed tracking section for outputting the tracking information about the subject's tissue based on the phase of the signal component that has passed through the second filter and by reference to the imperfect tracking information.

3. The ultrasonic diagnostic apparatus of claim 1, wherein the tissue tracking section includes: a plurality of tracking sections for tracking the motion of the subject's tissue on the basis of the phases to generate multiple pieces of tracking information; and a computing section for figuring out noise-reduced tracking information based on the multiple pieces of tracking information generated by the tracking sections.

4. The ultrasonic diagnostic apparatus of claim 3, wherein the computing section figures out at least one of the simple average and a weighted average of the multiple pieces of tracking information.

5. The ultrasonic diagnostic apparatus of claim 1, further comprising an amplitude measuring section for measuring the amplitude of at least one of the multiple signal components,
wherein if the amplitude is equal to or smaller than a predetermined value, the tissue tracking section generates the tracking information without using the signal component, of which the amplitude has been measured.

6. The ultrasonic diagnostic apparatus of claim 1, wherein the transmitting section generates a signal to be transmitted that drives the ultrasonic probe so as to produce an ultrasonic wave, in which at least one of the frequency bands of the filters is boosted.

7. The ultrasonic diagnostic apparatus of claim 1, further comprising a property evaluating section for evaluating an attribute property of the subject based on the tracking information.

8. The ultrasonic diagnostic apparatus of claim 2, wherein the first filter belongs to a frequency band that passes a fundamental wave component of the ultrasonic wave and the second filter belongs to a frequency band that passes an n^{th} harmonic component (where n is an integer that is equal to or greater than two) of the ultrasonic wave.

9. A method for controlling an ultrasonic diagnostic apparatus using the control section of the apparatus, the method comprising the steps of:
(A) generating a received signal based on a reflected wave by transmitting and receiving an ultrasonic wave with an ultrasonic probe, the reflected wave being produced by getting the ultrasonic wave reflected by a subject's tissue;
(B) extracting multiple signal components, belonging to mutually different frequency bands, from the received signal;
(C) detecting the respective phases of the multiple signal components; and
(D) tracking the motion of the subject's tissue on the basis of the phases, thereby outputting tracking information.

10. The method of claim 9, wherein the step (B) includes extracting a signal component with a first center frequency and another signal component with a second center frequency that is higher than the first center frequency,
wherein the step (D) includes the steps of
(D1) tracking the motion of the subject's tissue on the basis of the phase of the signal component that has the first center frequency and outputting imperfect tracking information and
(D2) outputting the tracking information about the subject's tissue based on the phase of the signal component that has the second center frequency and by reference to the imperfect tracking information.

11. The method of claim 9, wherein the step (D) includes the steps of
(D1) tracking the motion of the subject's tissue on the basis of the phases to generate multiple pieces of tracking information and
(D2) figuring out noise-reduced tracking information based on the multiple pieces of tracking information.

12. The method of claim 11, wherein the step (D2) includes figuring out at least one of the simple average and a weighted average of the multiple pieces of tracking information.

13. The method of claim 9, further comprising the step of (E) measuring the amplitude of at least one of the multiple signal components between the steps (C) and (D),
wherein if the amplitude is equal to or smaller than a predetermined value, the step (D) includes generating the tracking information without using the signal component, of which the amplitude has been measured.

14. The method of claim 9, wherein the step (A) includes transmitting an ultrasonic wave, in which at least one of the frequency bands of the filters is boosted, toward the subject.

15. The method of claim 9, further comprising the step of (F) evaluating an attribute property of the subject based on the tracking information.

16. The method of claim 9, wherein the signal component with the first center frequency includes a fundamental wave component of the ultrasonic wave and the signal component with the second center frequency includes an n^{th} harmonic component (where n is an integer that is equal to or greater than two) of the ultrasonic wave.
